(19) United States
(12) Patent Application Publication
(10) Pub. No.: US 2023/0414721 A1
(43) Pub. Date: Dec. 28, 2023

(54) MATERIALS AND METHODS FOR SENSITIZING TUMORS TO IMMUNE RESPONSE

(71) Applicant: University of Florida Research Foundation, Inc., Gainesville, FL (US)

(72) Inventors: Elias Sayour, Newberry, FL (US); Hector Ruben Mendez-Gomez, Gainesville, FL (US); Sadeem Qdaisat, Gainesville, FL (US); Brandon Wummer, Fort Lauderdale, FL (US); Duane A. Mitchell, Gainesville, FL (US)

(21) Appl. No.: 18/037,516

(22) PCT Filed: Nov. 17, 2021
(86) PCT No.: PCT/US21/59741

§ 371 Date: May 17, 2023

Related U.S. Application Data
(60) Provisional application No. 63/115,393, filed on Nov. 18, 2020.

(51) Int. Cl. A61K 38/21; A61K 39/395; A61P 35/00

(52) U.S. Cl. CPC A61K 38/212; A61K 39/3955; A61P 35/00

(57) ABSTRACT

Provided herein is a method of improving sensitivity of a tumor to a host immune response, the method comprising administering to a subject in need thereof a Type I interferon and an immune checkpoint inhibitor, thereby increasing sensitivity of the tumor to a host immune response.

### CROSS-REFERENCE TO RELATED APPLICATION

This application claims the benefit of priority to U.S. Provisional Patent Application No. 63/115,393, filed on Nov. 18, 2020, the disclosure of which is hereby incorporated by reference in its entirety.

### GOVERNMENT SUPPORT CLAUSE

This invention was made with government support under grant number K08 CA199224, awarded by the National Institutes of Health. The government has certain rights in the invention.

### FIELD OF THE INVENTION

The disclosure provides materials and methods for sensitizing tumors to attack by the immune system.

### BACKGROUND

Cancer therapy has been revolutionized through the introduction of immune checkpoints inhibitors (ICIs). By blocking the pathways that regulate the immune system, ICIs are capable of enhancing immunity, boosting T-cell activity, and reinitiating the recognition and destruction of cancer cells. To date, seven ICIs have been approved by the U.S. Food and Drug Administration, with more than 20 others currently being investigated and under clinical trials. See, e.g., Verma et al., Journal for Immunotherapy of Cancer. 2018; 6(1):128; Chauhan et al., Annals of oncology: official journal of the European Society for Medical Oncology. 2017; 28(8):2034-2038. However, many patients are resistant to immune checkpoint blockade, and little is understood regarding the mechanisms behind inherent or acquired resistance to ICIs. There is a need for treatment options for patients with limited response to ICIs.

### SUMMARY

The disclosure provides a method of improving sensitivity of a tumor to a host immune response. The method comprises administering to a subject in need thereof a Type I interferon and an immune checkpoint inhibitor, thereby increasing sensitivity of the tumor to a host immune response. In various aspects, the immune checkpoint inhibitor is a PD-1 inhibitor, such as an anti-PD-1 antibody. Optionally, the Type I interferon is IFN-alpha. In some embodiments, the Type I interferon is administered to the subject via intratumoral injection. In various aspects, tumor was refractory to immune checkpoint therapy prior to treatment. Optionally, the tumor is glioma.

### BRIEF DESCRIPTION OF THE DRAWINGS

FIGS. 1A-1C are graphs demonstrating that blockade of IFN-alpha during tumorigenesis abrogates activity from ICIs in immune sensitive models. FIG. 1A: The graph summarizes Kaplan-Meier survival analysis of mortality events for GL261 mice of different treatment groups (i.e., untreated, PD1 mAbs+IFNAR1 mAbs combination, or PD-1 mAbs alone) with day 1 as a starting treatment date followed by biweekly injection from day 2 to day 21. Percent survival (y-axis) is provided over days after tumor implantation (x-axis). Circles represent untreated subjects; squares represent subjects administered anti-PD-1 antibodies and anti-INFAR1 antibodies; triangles represent subjects administered anti-PD-1 antibodies alone. FIG. 1B: The graph summarizes Kaplan-Meier survival analysis of mortality events for GL261 mice of different treatment groups (i.e., untreated, PD1 mAbs+IFNAR1 mAbs combination, or PD-1 mAbs alone) with day 5 as a starting treatment date followed by biweekly injection from day 6 to day 21. FIG. 1C: The graph summarizes that myeloid cells are necessary for response to checkpoint inhibitors. These cells are responsible for IFN-alpha release and T cell priming necessary for checkpoint inhibitor responsiveness.

FIGS. 2A-D are graphs demonstrating that early blockade of IFN-alpha enables increased tumorigenicity of immunogenic cancers. FIGS. 2A-2B: Percentage of mice with visible tumors (A) and absolute count of nodules in each lung (B) two weeks after inoculation with 150,000 B16F10-OVA cells with or without IFNAR1 blocking antibodies administered 24 hours before and after tumor injection, then biweekly until tumor harvest. FIG. 2C: Tumor growth measurements for subcutaneous B16F10-OVA tumors with or without blockade of IFNAR1 starting upon implantation (left) or at 5 days after implantation (right). FIG. 2D: Growth of B16F0 tumors with or without blockade of IFNAR1.

FIGS. 3A-E illustrate that sensitivity to immune checkpoint inhibitors in responsive models is transferrable to resistant models in an IFNAR1 dependent manner FIG. 3A: Diagram of experimental design. Mice were inoculated with contralateral, tumorigenic doses of B16F10-OVA and B16F0 and treated with either no treatment or PD-1 inhibition with or without IFNAR1 blockade starting one day after tumor implantation. FIG. 3B: Tumor growth curve for ICI-resistant B16F0 tumors. FIG. 3C: Donor mice were inoculated with B16F10-OVA cells and treated with biweekly PD-1 mAbs alone or in combination with early IFNAR1 blockade. Cd3+ splenocytes were harvested from mice in each treatment group 20 days after tumor implantation and transferred to recipient mice who were inoculated 24 hours previously with ICI-resistant B16F0 cells. These recipient mice were then treated with biweekly PD-1 mAbs. FIG. 3D: Tumor growth curves comparing outcomes for mice receiving PD-1 therapy alone or in combination with CD3+ cells from sensitized mice or those treated with early IFNAR1 blockade.

FIG. 3E: Kaplan-Meier survival curves comparing outcomes for mice receiving PD-1 therapy alone or in combination with CD3+ cells from sensitized mice or those treated with early IFNAR1 blockade (**p<0.01, ***p<0.001).

FIGS. 4A-C illustrate that immunogenicity may be restored with intratumoral IFN-alpha administration. FIG. 4A: Kaplan-Meier survival curve for mice bearing B16F0 cells treated with PD-1 only or admixed with IFN-alpha (IFN-alpha+PD1). Untreated controls without PD-1 therapy are included for comparison (Untreated). FIG. 4B: Tumor growth curve for mice in FIG. 4A. FIG. 4C: Tumor growth for mice with subcutaneous B16F0 tumors with or without PD-1 blockade and systemic administration of IFN-alpha.

### DETAILED DESCRIPTION

There remains a need for novel immune potentiating approaches that can increase the effectiveness of the host immune system against cancer cells, especially in view of the prevalence of patients that do not respond well (or at all) to immunotherapy.

The disclosure provides a method of improving sensitivity of a tumor to a host immune response. The method comprises administering to a subject in need thereof a Type I interferon and an immune checkpoint inhibitor (ICI), thereby increasing sensitivity of the tumor to a host immune response. As described further below, it has now been determined that Type I IFN (e.g., IFN-alpha) signaling during tumorigenesis is requisite for future responsiveness to ICI, and administration of IFN-alpha restores immune sensitization of resistant tumors. Thus, the method described herein improves the sensitivity of tumors to a host immune response, providing an advancement in the field of cancer immunotherapy.

Type I interferons (IFNs) are a large subgroup of proteins that bind to the cell surface receptor complex, IFN-α receptor (IFNAR). Type I IFNs play a role in activating key components of both the innate and adaptive immune systems, including antigen presentation and production of cytokines involved in activation of T cells, B cells, and natural killer cells. Type I interferons include IFN-alpha, IFN-beta, IFN-kappa, IFN-delta, IFN-epsilon, IFN-tau, IFN-omega, and IFN-zeta (also referred to as limitin). See, e.g., Platanias, Nat Rev Immunol. (2005) 5:375-86. In various aspects, the Type I interferon is IFN-alpha. Multiple subtypes of IFN-alpha exist, including IFN-alpha1, IFN-alpha2, IFN-alpha8, IFN-alpha10, IFN-alpha14, and IFN-alpha21. Several IFN-alpha products have been approved for use in humans by regulatory agencies. Examples of IFN-alpha products include, but are not limited to, Intron® A (interferon alfa-2b), Roferon®-A (interferon alfa-2a), Alferon-N® (interferon alfa-n3), as well as PegIntron™ and Sylatron™ (peginterferon alfa-2b).

The method described herein comprises administering an immune checkpoint inhibitor (ICI) to the subject. Optionally, two or more ICIs may be administered. The term “immune checkpoint inhibitor” refers to a molecule or therapeutic that decreases, blocks, inhibits, abrogates or interferes with the function of a protein of an immune checkpoint pathway. Proteins of the immune checkpoint pathway regulate immune responses and, in some instances, prevent T cells from attacking cancer cells. In some embodiments, the ICI is a small molecule, an inhibitory nucleic acid, an inhibitory polypeptide, or an antibody or antigen-binding domain thereof. In various aspects, the ICI targets a protein in an immune checkpoint pathway to reduce expression or inhibit activity. Proteins in the immune checkpoint pathway include, but are not limited to, PD-1, PD-L1, PD-L2, CD28, CTLA-4, B7-H3, B7-H4, B7-1, and B7-2 (see National Cancer Institute Dictionary of Cancer Terms), as well as ICOS, BTLA, TIM3, VISTA, TIGIT, and LAG3. In some embodiments, the checkpoint inhibitor is an antibody or antigen-binding domain thereof that binds an immune checkpoint polypeptide (e.g., PD-1, PD-L1, CTLA4, or PD-L2) and inhibits its activity.

In various aspects, the ICI is an antibody or antigen-binding fragment thereof. As used herein, the term “antibody” refers to a protein having a conventional immunoglobulin format, comprising heavy and light chains, and comprising variable and constant regions. The general structure and properties of antibodies have been described in the art. Briefly, in an antibody scaffold, complementarity determining regions (CDRs) are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra). Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′) 2 fragment and a Fc fragment. Optionally, antigen binding antibody fragment is incorporated into a fusion protein. An antigen binding antibody fragment refers to a portion of an antibody molecule that is capable of binding to the antigen of the antibody. In exemplary instances, the antigen binding antibody fragment is a Fab fragment or a F(ab′)₂ fragment.

Inhibitors of checkpoint regulators (e.g., PD-L1, PD-L2, PD-1, CTLA-4, TIM-3, LAG-3, VISTA, or TIGIT) are known in the art. Non-limiting examples of ICIs with corresponding checkpoint targets include: MGA271 (B7-H3: MacroGenics); ipilimumab (CTLA-4; Bristol Meyers Squibb); pembrolizumab (PD-1; Merck); nivolumab (PD-1; Bristol Meyers Squibb); atezolizumab (PD-L1; Genentech); IMP321 (LAG3 Immuntep); BMS-986016 (LAG3; Bristol Meyers Squibb); IPH2101 (KIR; Innate Pharma); tremelimumab (CTLA-4; Medimmune); pidilizumab (PD-1; Medivation); MPDL3280A (PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); and TSR-022 (TIM3; Tesaro).

In some embodiments, the checkpoint inhibitor inhibits PD-1. “Programmed Death-1” (PD-1), also known as cluster of differentiation 279 (CD279), refers to an immunoinhibitory receptor belonging to the CD28 family. PD-1 is expressed on previously activated T cells in vivo, and binds to two ligands, PD-L1 and PD-L2. The human PD-1 sequence can be found under GenBank Accession No. U64863. In another embodiment, the checkpoint inhibitor inhibits PD-L1. Programmed death-ligand 1 (PD-L1; also known as cluster of differentiation 274 (CD274) or B7 homolog 1 (B7-H1)) is a transmembrane protein that functions to suppress the immune system in, e.g., pregnancy, tissue allografts, and autoimmune disease. Binding of PD-L1 to its receptor PD-1 transmits an inhibitory signal that reduces the proliferation and function of T cells and can induce apoptosis. PD-L1/PD-1 blockade can be accomplished by a variety of mechanisms, including antibodies that bind PD-1 or PD-L1.

Examples of PD-1 and PD-L1 inhibitors are described in, e.g., U.S. Pat. Nos. 7,488,802; 7,943,743; 8,008,449; 8,168,757; 8,217,149: and PCT Patent Publication Nos. WO03042402, WO2008156712, WO2010089411, WO2010036959, WO2011066342, WO2011159877, WO2011082400, and WO2011161699; which are incorporated by reference herein in their entireties. PD-1 inhibitors include anti-PD-1 antibodies and similar binding proteins such as nivolumab, a fully human IgG4 antibody that binds to and blocks the activation of PD-1 by its ligands PD-L1 and PD-L2; lambrolizumab, a humanized monoclonal IgG4 antibody against PD-1; pidilizumab, a humanized antibody that binds PD-1; pembrolizumab (Keytruda™), a humanized monoclonal IgG4 kappa antibody against PD-1; AUNP-12, a small branched peptide inhibitor of the PD-1/PD-L1 pathway further described in International Patent Publication No. WO2011161699; and AMP-224, a fusion protein comprising B7-DC. PD-L1 inhibitors include, but are not limited to atezolizumab, an Fc-engineered, humanized, non-glycosylated IgG1 kappa immunoglobulin that targets PD-L1; avelumab, a human IgG1 lambda monoclonal antibody against PD-L1, and durvalumab, a human IgG1 kappa monoclonal antibody against PD-L1.

Cytotoxic T-lymphocyte-associated protein 4 (CTLA-4, also known as CD152), is a membrane protein expressed on T cells and regulatory T cells (Treg). CTLA-4 binds B7-1 (CD80) and B7-2 (CD86) on antigen-presenting cells (APC), which inhibits the adaptive immune response. In humans, CTLA-4 is encoded in various isoforms; an exemplary amino acid sequence is available as GenBank Accession No. NP_001032720. A representative anti-CTLA-4 antibody is ipilimumab (YERVOY®, Bristol-Myers Squibb).

The disclosure provides a method of improving sensitivity of a tumor to a host immune response. “Sensitivity” refers to the way a tumor reacts to a therapeutic, e.g., a ICI inhibitor (e.g., PD-1 inhibitor) or a host immune response. In exemplary aspects, “sensitivity” means “responsive to treatment” and the concepts of “sensitivity” and “responsiveness” are positively associated in that a tumor or cancer cell that is responsive to stimuli (e.g., a treatment) is said to be sensitive to that stimuli (treatment). “Sensitivity,” in exemplary, illustrative instances, is defined according to Pelikan, Edward, Glossary of Terms and Symbols used in Pharmacology (Pharmacology and Experimental Therapeutics Department Glossary at Boston University School of Medicine), as the ability of a population, an individual, or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistant” and the concept of “resistance” is negatively associated with “sensitivity.” For example, a tumor that is resistant to a treatment is neither sensitive nor responsive to that treatment, and that treatment is not an effective treatment for that tumor or cancer cell. “Sensitivity” also is used herein with respect to a host immune response. In this respect, a tumor which evades a host immune response is “resistant” (or refractory). A tumor that is “sensitive” to a host immune response is recognized by the host immune system and subject to attack by immune effector cells. In the context of the disclosure, administration of a Type I interferon sensitizes a tumor to an ICI, and together the two active agents increase the sensitivity of the tumor to a host immune response. An increase in sensitivity provided by the methods of the present disclosure may be at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative to a control. The increase in sensitivity provided by the methods of the present disclosure may be at least or about a 10% to greater than about a 95% increase (e.g., at least or about a 10% increase, at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase, at least or about a 100% increase) relative to a control. In exemplary aspects, the control is cancer or tumor or a subject or a population of subjects that was not treated with the presently disclosed method or wherein the subject or population of subjects was treated with a placebo.

Increased sensitivity to an ICI or increased sensitivity to host immune response may be determined in any of a number of ways. For example, administration of the Type I IFN and ICI may increase the number of cytotoxic T cells in a tumor and/or enhance cytotoxic T cell activity. For example, in various embodiments, the method may increase perforin, IFN-gamma, and/or granzyme production by cytotoxic T cells and increase cytolytic activity. Further, the method described herein may enhance T cell survival, promote T cell longevity, and/or restrict loss of replicative potential. Methods of measuring T cell activity and immune responses are known in the art. T cell activity can be measured by, for example, a cytotoxicity assay, such as those described in Fu et al., PLoS ONE 5(7): e11867 (2010). Other T cell activity assays are described in Bercovici et al., Clin Diagn Lab Immunol. 7(6): 859-864 (2000). Methods of measuring immune responses are described in e.g., Macatangay et al., Clin Vaccine Immunol 17(9): 1452-1459 (2010), and Clay et al., Clin Cancer Res. 7(5):1127-35 (2001). In various aspects, the method of the disclosure enhances cytotoxic T cell mediated killing of cancer cells within the tumor.

In various aspects, the tumor is refractory to immune checkpoint therapy prior to administration of the Type I IFN, i.e., one or more ICIs has reduced efficacy in eliciting an immune response against the tumor. Alternatively, the tumor is not refractory, but the method further enhances sensitivity to the immune response such that enhanced tumor cell death is achieved.

In exemplary embodiments, the method comprises administering to the subject a Type I IFN and an ICI in amounts effective for treating the cancer or the tumor (e.g., solid tumor) in the subject. The cancer treatable by the methods disclosed herein can be any cancer, e.g., any malignant growth or tumor caused by abnormal and uncontrolled cell division. The cancer in some aspects is acute lymphocytic cancer, acute myeloid leukemia, alveolar rhabdomyosarcoma, bone cancer, brain cancer (e.g., glioma), breast cancer (e.g., triple negative breast cancer), cancer of the anus, anal canal, or anorectum, cancer of the eye, cancer of the intrahepatic bile duct, cancer of the joints, cancer of the head, neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal cancer (e.g., gastrointestinal carcinoid tumor), gastric cancer, Hodgkin lymphoma, hypopharynx cancer, endometrial or hepatocellular carcinoma, kidney cancer, larynx cancer, liver cancer, lung cancer (e.g., non-small cell lung cancer (NSCLC), bronchioloalveolar carcinoma), malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, colorectal cancer, thyroid cancer, ureter cancer, or urinary bladder cancer. In various aspects, the subject has a solid tumor. Optionally, the subject suffers from a malignant brain tumor, such as a glioblastoma, medulloblastoma, diffuse intrinsic pontine glioma, or a peripheral tumor with metastatic infiltration into the central nervous system.

As used herein, the term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating cancer or treating a subject with a solid tumor can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, thereby reducing tumor or cancer growth, reducing metastasis of tumor cells, increasing cell death of tumor or cancer cells, and the like.

The susceptibility of a tumor to an immune response or, put another way, the effectiveness of an immune response against a tumor, can be determined in a variety of ways, including detection of a change in tumor mass and/or volume after treatment. For example, the size of a tumor may be compared to the initial size and dimensions as measured by CT, PET, mammogram, ultrasound, or palpation, as well as by caliper measurement or pathological examination of the tumor after biopsy or surgical resection. Response may be characterized quantitatively using, e.g., percentage change in tumor volume (e.g., the method of the disclosure results in a reduction of tumor volume by at least 10%, at least 20%, at least 30%, at least 40%, at least 50%, at least 60%, at least 70%, at least 80%, or at least 90%). Alternatively, tumor response or cancer response may be characterized in a qualitative fashion like “pathological complete response” (pCR), “clinical complete remission” (cCR), “clinical partial remission” (cPR), “clinical stable disease” (cSD), “clinical progressive disease” (cPD), or other qualitative criteria. In addition, treatment efficacy also can be characterized in terms of responsiveness to other immunotherapy treatment or chemotherapy. Efficacy of treatment also can be characterized in terms of cancer biomarker prevalence.

The subject of the disclosure is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammal is of the order Primate, Ceboid, or Simoid (monkeys) or of the order Anthropoid (humans and apes). In some aspects, the mammal is a human. A subject may be one who has been previously diagnosed with or identified as suffering from or having a condition in need of treatment (e.g., cancer) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition. The subject, in various aspects, has previously received a treatment or therapy for the condition (e.g., previously been administered an anti-cancer therapy).

The active agents described herein (Type I IFN and ICI) can be administered to the subject via any suitable route of administration. For example, the active agents can be administered to a subject via parenteral, nasal, oral, pulmonary, topical, vaginal, or rectal administration. Parenteral dosage forms of an agent described herein can be administered to a subject by various routes, including, but not limited to, epidural, intracerebral, intracerebroventricular, epicutaneous, intraarterial, intraarticular, intracardiac, intracavernous injection, intradermal, intralesional, intramuscular, intraocular, intraosseous infusion, intraperitoneal, intrathecal, intrauterine, intravaginal administration, intravenous, intravesical, intravitreal, subcutaneous, transdermal, perivascular administration, or transmucosal. For administration to the brain, a pharmaceutical composition can be introduced into tumor tissue using an intratumoral delivery catheter, ventricular shunt catheter attached to a reservoir (e.g., Omaya reservoir), infusion pump, or introduced into a tumor resection cavity (such as Gliasite, Proxima Therapeutics). Tumor tissue in the brain also can be contacted by administering a pharmaceutical composition via convection using a continuous infusion catheter or through cerebrospinal fluid.

In various aspects, the Type I IFN is locally administered (within close proximity) to the tumor. For example, the Type I IFN is directly instilled into a cavity were the tumor is located or introduced into an artery feeding the tumor. In this regard, the Type I IFN may also be administered intratumorally (i.e., injection or instillation directly into the tumor). A dose may be delivered to a tumor via multiple applications (injections) to different points of the target tumor, although this is not required. Multiple applications can be manipulated by such parameters as a specific geometry defined by the location on the tumor where each application is administered to ensure that a single dose is uniformly distributed throughout the tumor. In various embodiments, the ICI is administered locally, intratumorally, or intravenously.

Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions. Suitable vehicles for therapeutic dosage forms are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed., pages 622-630 (1986)).

The active agents of the disclosure are useful in methods of sensitizing tumors to immunotherapy and, thus, are believed to be useful in methods of treating or preventing one or more diseases, e.g., cancer. For purposes of the disclosure, the amount or dose of the active agent (i.e., the “effective amount”) administered should be sufficient to achieve a desired biological effect, e.g., a therapeutic or prophylactic response, in the subject over a reasonable time frame. For example, one or more doses of the Type I IFN and ICI of the present disclosure should be sufficient to, e.g., sensitize a tumor to an immune response (and optionally treat a cancer) in a clinically acceptable period of time e.g., 1 to 20 or more weeks, from the time of first administration. In certain embodiments, the time period could be even longer. The dose will be determined by the efficacy of the particular active agents, the condition of the animal (e.g., human), as well as the body weight of the animal (e.g., human) to be treated, and the existence, nature and extent of any adverse side effects that might accompany the administration of a particular active agent. By way of example and not intending to limit the present disclosure, the dose of the active agents of the present disclosure can be about 0.0001 to about 1 g/kg body weight of the subject being treated/day, from about 0.0001 to about 0.001 g/kg body weight, or about 0.01 mg to about 1 g/kg body weight.

In some embodiments, the method described herein further comprises administration of one or more other therapeutic agents. In some aspects, the other therapeutic agent aims to treat or prevent cancer. In some embodiments, the other therapeutic is a chemotherapeutic agent. Common chemotherapeutics include, but are not limited to, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, rituximab, streptozocin, teniposide, thioguanine, thiotepa, vinblastine, vincristine, vinorelbine, taxol, transplatinum, 5-fluorouracil, and the like. In some embodiments, the other therapeutic is an agent used in radiation therapy for the treatment of cancer; indeed, in some embodiments, the method is part of a treatment regimen that includes radiation therapy.

Further, the method of the disclosure can be performed in connection with surgical resection of a tumor, such as a glioma (e.g., glioblastoma). Complete surgical removal of tumor tissue is often complicated by invasion of the tumor tissue into surrounding tissues and indefinite margins of the mass. Treatment of a tumor using the method described herein leads to tumor shrinkage, which facilitates resection. Moreover, the method of the disclosure, when performed post-surgery, can eliminate residual tumor cells. As such, in various aspects of the disclosure, the method comprises instilling IFN-alpha into a resection cavity, in conjunction with administration of an ICI (either also instilled into the resection cavity or administered systemically). In any embodiment described herein, the IFN-alpha and ICI may be administered together (in the same formulation or separate formulations administered close in time) or may be administered sequentially (i.e., the IFN-alpha is administered and the ICI is administered separately at different time points (e.g., hours or days apart)).

The present disclosure additionally provides kits comprising an immune checkpoint inhibitor (e.g., a PD-1 antigen-binding protein, such as an anti-PD-1 antibody) and IFN-alpha in containers with instructions for use. In exemplary aspects, the checkpoint inhibitor and IFN-alpha are provided in the kit as unit doses. “Unit dose” refers to a discrete amount dispersed in a suitable carrier. In exemplary aspects, the unit dose is the amount sufficient to provide a subject with a desired effect, e.g., cancer cell death. In exemplary aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, components of the kit are pre-packaged in a ready to use form, e.g., a syringe, an intravenous bag, etc. In exemplary aspects, the ready to use form is for a single use. In exemplary aspects, the kit comprises multiple single use, ready to use forms of the components. In some aspects, the kit further comprises other therapeutic or diagnostic agents or pharmaceutically acceptable carriers (e.g., solvents, buffers, diluents, etc.), including any of those described herein.

It will be appreciated that the disclosure provides use of a Type I interferon to improve the sensitivity of a tumor to a host immune response or sensitize a tumor to an ICI, wherein the Type I interferon and an ICI is administered to the subject. The disclosure provides use of a Type I interferon to improve the sensitivity of a tumor to ICI therapy, wherein the Type I interferon and an ICI is administered to the subject. The disclosure provides use of a Type I interferon and an immune checkpoint inhibitor to improve sensitivity of a tumor to a host immune response (thereby treating the cancer or a tumor). The disclosure further provides a Type I interferon and an immune checkpoint inhibitor for use in a method of improving sensitivity of a tumor to a host immune response (thereby treating a subject for cancer). In various aspects, the immune checkpoint inhibitor is a PD-1 inhibitor, such as an anti-PD-1 antibody. In various aspects, the tumor was refractory to immune checkpoint therapy prior to treatment or immunologically “cold” (e.g., e.g., a tumor lacking infiltrating T cells and/or which is not recognized by the immune system). In various aspects, the tumor is glioma. In various aspects, the Type I interferon is IFN-alpha. Optionally, the Type I interferon is administered to the subject via intratumoral injection and/or the subject has undergone surgical resection of a tumor and the IFN-alpha is administered to the resection cavity.

### EXAMPLES

The following example is given merely to illustrate the present disclosure and not in any way to limit its scope.

### Example 1

This example demonstrates that administration of IFN-alpha restores immune sensitivity to resistant tumors.

### Materials and Methods

Cell Culture: Tumor cell lines B16-F0, B16F10-OVA, and GL-261 were obtained as previously described. B16F0 is a murine melanoma cell line purchased from ATCC. B16F10-OVA is a murine melanoma cell line transfected with the chicken ovalbumin gene (OVA). B16-F0 and B16F10-OVA were cultured in DMEM with pyruvate containing 10% fetal bovine serum (FBS) and 1% penicillin/streptomycin at conditions of 37° C. with 5% CO 2 levels.

Immunohistochemistry: Lymph nodes were harvested and suspended overnight in 4% buffered paraformaldehyde solution at 4° C. The nodes were washed three times in phosphate-buffered saline (PBS) before overnight immersion in 15% sucrose at 4° C., followed by overnight immersion in 30% sucrose at 4° C. The nodes were then submerged in Tissue-Tek OCT (Electron Microscopy Sciences, Hatfield, PA), frozen in liquid nitrogen, and stored at −80° C. until sectioning. Sections of 10 μm thickness were cut on a Leica CM1850 cryostat (Leica Microsystems) and stored at room temperature until immunohistochemistry (IHC) processing.

Tumor Implantation: B16F0, B16F10-OVA, and GL-261 cells were harvested from culture with 0.05% trypsin (Gibco) and washed in serum-containing PBS before being washed in PBS. Cell pellets were resuspended in PBS at a concentration of 107 cells/mL. Subcutaneous—B16F0 and B16F10-OVA cells were injected subcutaneously using a 25-gauge syringe into the right flank of C57Bl/6 mice anesthetized with isoflurane. Subcutaneous tumors were measured three times weekly with a Westward digital caliper and their volumes calculated by multiplying the greatest longitudinal (length), transverse (width), and vertical (height) components of each tumor. Intracranial—The GL-261 cells were resuspended in a mixture of PBS:methyl cellulose mixture (at a 1:1 ratio) at 8 million cells/mL before stereotactic intracranial implantation (2 mm to the right of the bregma and 3 mm deep into the brain) using a Hamilton Microliter Syringe.

Mice: C57Bl/6 mice were purchased from Jackson Laboratories

Checkpoint Inhibition: Anti-PD-1 (clone: RMP1-14) and anti-PD-L1 (clone: 10F.9G2) monoclonal antibodies were purchased from BioXcell and administered into the peritoneum (IP) of C57Bl/6 mice using a 400 μg loading dose followed by 200 μg twice weekly. IFN-alpha blocking monoclonal antibodies (clone: MAR1-5A3) were purchased from BioXcell and administered IP using a 500 μg loading dose followed by 250 μg twice weekly. All injections continued for three weeks following the first treatment.

IFN-α ELISAs: C57Bl/6 mice were bled retro-orbitally and their serum separated through coagulation and centrifugation. Detection of IFN-alpha was assessed using ELISA purchased from Thermo Fischer (#BMS6027). For the assay, 25 μL of serum were used instead of the recommended 50 μL.

Macrophage Depletion: To selectively deplete macrophages in vivo, clodronate liposomes were purchased from Liposome and intravenously injected into the tail vein of C57Bl/6 mice with a loading dose of 200 μL. Macrophage depletion occurred the day prior to intracranial tumor implantation.

### Results

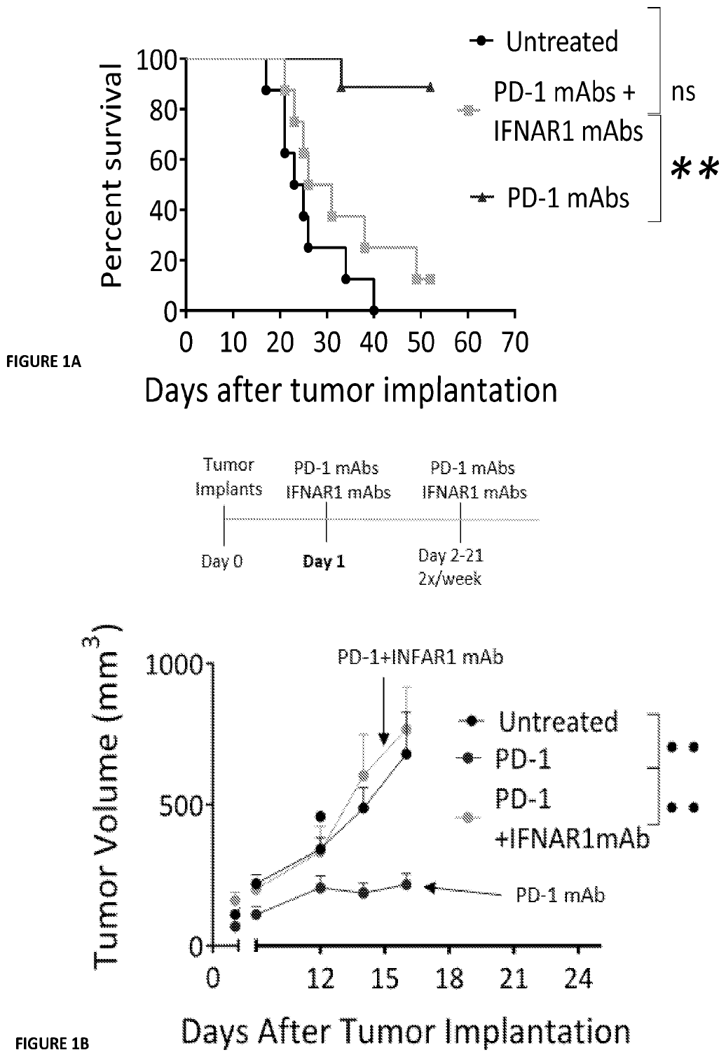

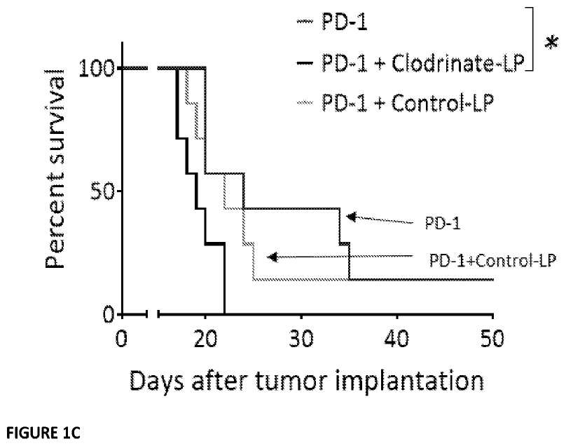

Early Blockade of IFN-alpha removes susceptibility to ICI: Although early IFN-alpha production enhances antitumor adaptive immune function, late IFN-alpha expression abrogates antitumor immune responses to ICIs, which function via adaptive immunity. Based on these paradoxical findings, it was hypothesized that early secretion of IFN-alpha at the tumor site is a powerful driver of response to ICI. The role of IFN-alpha production in tumorigenesis and responsiveness to ICI was examined A series of experiments were conducted with ICI-sensitive tumor models (B16F10-OVA and GL261) and ICI-resistant tumors (B16F0). First, mice were treated with biweekly systemic blockade of PD-1 and IFN-alpha receptor 1 (IFNAR1) starting just one day after intracranial implantation of GL261, a murine model of glioma with established sensitivity to PD-1 blockade. Although early administration of PD-1 starting on Day 1 after tumor implantation significantly enhanced survival, producing 90% long term survivors, concomitant administration of IFNAR1 blocking antibodies completely abrogated this response, indicating that early IFN-alpha signaling was essential for the response to checkpoint inhibition in this model (FIG. 1A). The time dependence of the necessity of IFN-alpha signaling was then evaluated. Mice were treated with combination therapy of PD-1 and IFNAR1 blockade starting on Day 5 after tumor implantation. In this setting, the addition of IFN-alpha blockade had no effect on PD1-induced suppression of tumor growth in both GL261 and B16F10-OVA, indicating that the necessity of IFN signaling had waned by 5 days after tumor implantation (FIG. 1B). In order to isolate the impact of early IFN-alpha stimulus, a third group of mice was treated with IFN-alpha blockade during tumorigenesis (Days 1, 3, and 5) and withheld PD-1 blockade until day 5. As in the first experiment, this early initiation of IFN-alpha blockade precluded benefit of later therapy with PD-1. Taken together, this data suggests that IFN-alpha signaling during tumorigenesis is requisite for future responsiveness to ICI.

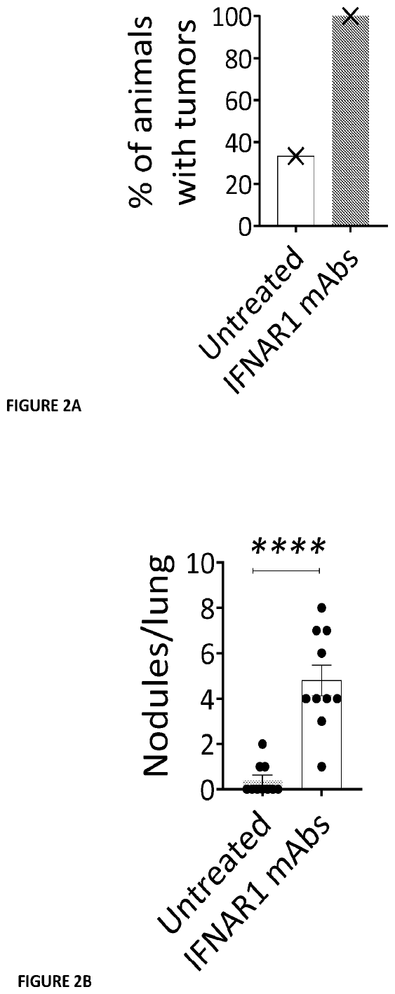

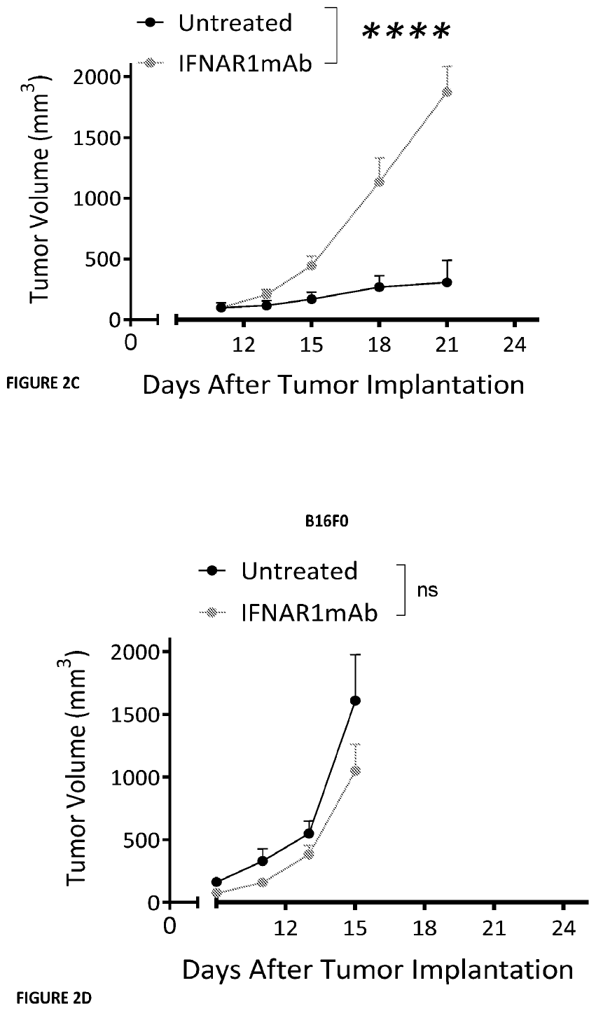

Early IFN-alpha signaling reduces tumorigenicity of immunogenic cancers: Given the importance of early IFN-alpha signaling in generation of checkpoint-mediated antitumor immune responses, the importance of IFN-alpha signaling on tumorigenesis was investigated in the absence of checkpoint blockade. B16F10-OVA cells were injected intravenously to simulate metastatic tumor seeding, and the dose for tumorigenicity was titrated. Concomitant administration of IFNAR1 blocking antibodies enabled uniform tumor formation after a tumor cell inoculation that produces tumors in only 30% of untreated mice (FIG. 2A). Furthermore, the untreated mice that did form tumors had fewer nodules per lung than those treated with IFN-alpha blockade (FIG. 2B), suggesting that early IFN-alpha signaling allows for immune surveillance and regulation of tumor growth. These findings were then tested with subcutaneous tumor models. In this setting, immunogenic B16F10-OVA tumors grew significantly larger in the setting of early IFN-alpha blockade but were unaffected by late blockade (FIG. 2C). FIG. 2D illustrates growth of B16F0 tumors with or without blockade of IFNAR1.

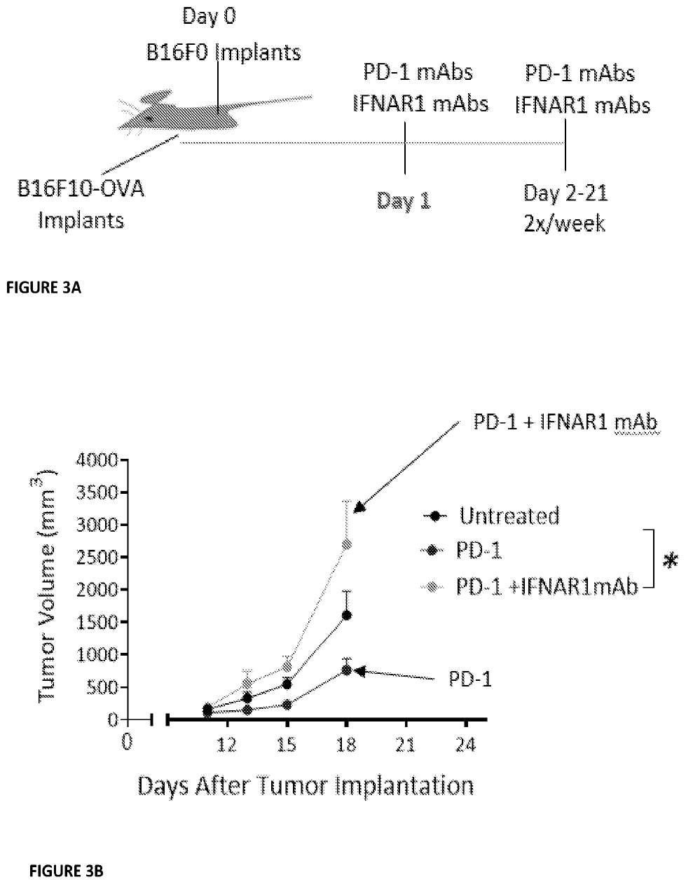

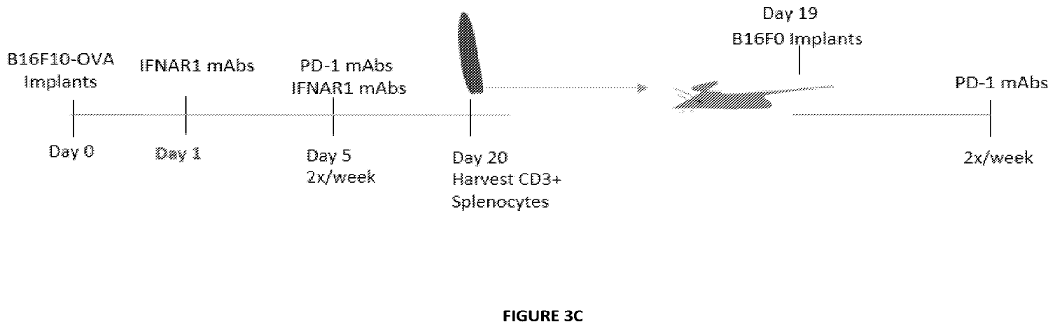

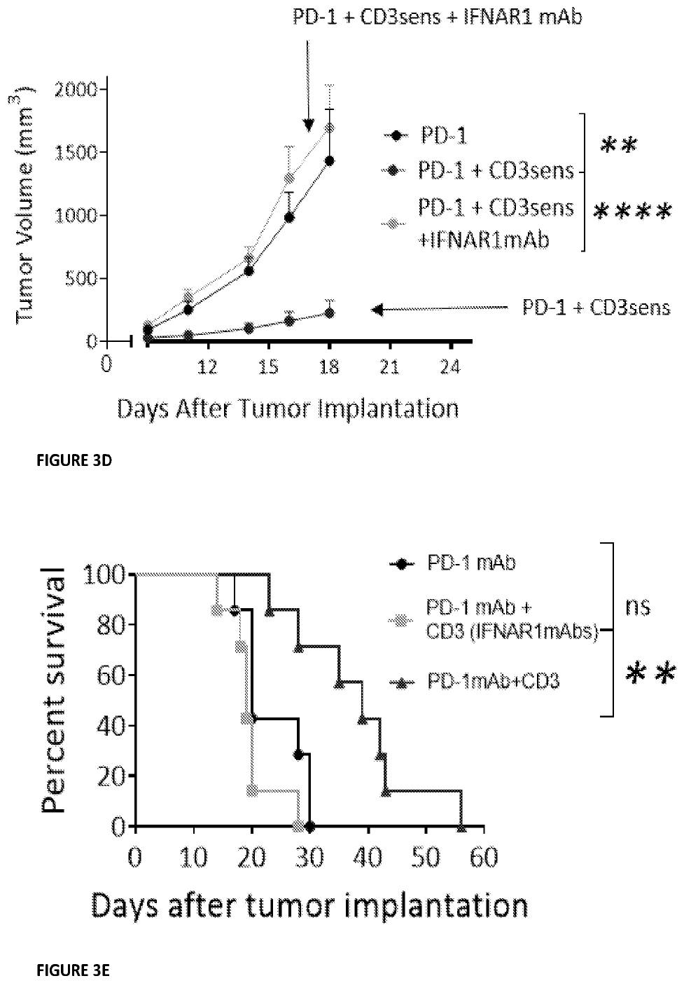

Sensitivity to ICIs is maintained by CD3+ cells that can be transferred to resistant tumor models: The impact of Type I IFN signaling on distant tumors was exampled. To begin, a model was designed in which an immune response to ICI resistant tumors could be evaluated. Mice with both an ICI-sensitive tumor (B16F10-OVA) and an ICI-resistant tumor (B16F0) with shared antigens (FIG. 3A) were inoculated. In this case, ICI with PD-1 blockade resulted in inhibition of growth of the ICI-resistant tumor (FIG. 3B). However, addition of early blockade of Type I IFN signaling abrogated this effect. This evidence suggests that IFN-alpha signaling is necessary for ICI-sensitive tumors to impart sensitivity to ICI-resistant tumors. Whether this sensitivity was mediated through innate or adaptive immune cells was explored. To this end, mice with ICI sensitive tumors (B16F10-OVA) were treated to achieve PD-1 blockade in the presence or absence of early IFN-alpha signaling; then CD3+ cells were transferred from these mice to a group of mice with ICI resistant tumors with shared tumor antigens (B16F0) (FIG. 3C). T cells from mice treated with PD-1 blockade were able to initiate potent rejection of ICI-resistant tumors in recipient mice leading to significant inhibition of tumor growth and prolonged survival in these mice (FIGS. 3D-E). However, T cells from donor mice treated with early IFN-alpha blockade imparted no benefit. This data suggests that sensitivity to ICI is dependent on early IFN-alpha signaling and mediated by long-term activation of CD3+ cells.

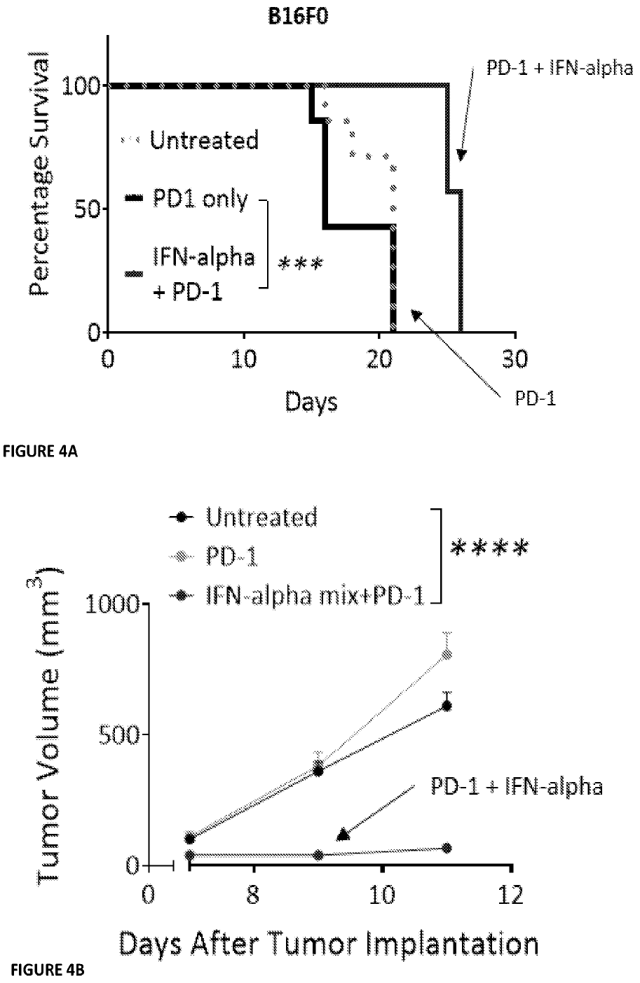

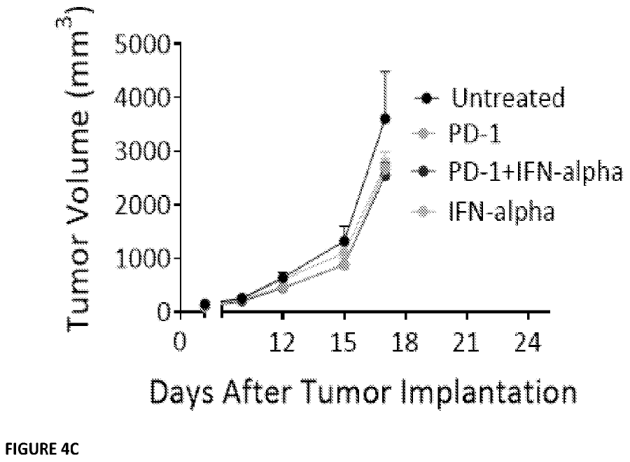

Intratumoral IFN-alpha restores immune sensitization of resistant tumors: Given this finding, it was hypothesized that sensitivity to ICI could be imparted with early introduction of IFN-alpha at the tumor site. To evaluate this hypothesis, mice were implanted with ICI-resistant B16F0 cells in medium containing IFN-alpha, then treated to achieve PD-1 inhibition. The addition of IFN-alpha during tumorigenesis was sufficient to significantly extend survival by over 30% compared to either no treatment or non-sensitized cells (FIG. 4A). Mice receiving B16F0 cells mixed with IFN-alpha also exhibited significantly reduced tumor growth (FIG. 4B). To verify this result, it was confirmed that this effect could also be achieved without direct injection of IFN-alpha by culturing tumor cells with IFN-alpha before implantation (FIG. 4B). The location of IFN-alpha signaling that drove this effect was evaluated. Mice were treated with systemic injections of IFN-alpha on the day of tumor implantation. Surprisingly, this systemic administration of IFN-alpha had not impact on response to ICI (FIG. 4C). Taken together, this data suggests that early, localized IFN-alpha production at the tumor site drives sensitivity to ICI.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of improving sensitivity of a tumor to a host immune response, the method comprising administering to a subject in need thereof a Type I interferon and an immune checkpoint inhibitor, thereby increasing sensitivity of the tumor to a host immune response.

2. The method of claim 1, wherein the immune checkpoint inhibitor is a PD-1 inhibitor.

3. The method of claim 1 or claim 2, wherein the immune checkpoint inhibitor is an anti-PD-1 antibody.

4. The method of any one of claims 1-3, wherein the tumor was refractory to immune checkpoint therapy prior to treatment.

5. The method of any one of claims 1-4, wherein the tumor is glioma.

6. The method of any one of claims 1-5, wherein the Type I interferon is IFN-alpha.

7. The method of any one of claims 1-6, wherein the Type I interferon is administered to the subject via intratumoral injection.

8. The method of any one of claims 1-7, wherein the subject has undergone surgical resection of a tumor and the IFN-alpha is administered to the resection cavity.
